(19) United States
(12) Patent Application Publication
(10) Pub. No.: US 2011/0064789 A1
(43) Pub. Date: Mar. 17, 2011

(54) DOCOSAHEXANOIC ACID AS INHIBITOR OF H. PYLORI

(76) Inventors: Eliette Touati, Chatillon (FR); Marta Correia, Porto (PT); José Carlos Machado, Sandim (PT); Valérie Michel, Ozoir La Ferriere (FR)

(21) Appl. No.: 12/558,344

(22) Filed: Sep. 11, 2009

(51) Int. Cl. A61K 9/127; A61K 31/202; A61P 31/04

(52) U.S. Cl. 424/450; 514/560

(57) ABSTRACT

The present invention concerns a method for preventing, modulating or curing in a mammal, the infections with Helicobacter pylori or the symptoms associated with H. pylori infection(s) or related diseases, and/or improving the clinical condition of a mammal infected with H. pylori, or affected by a related disease, comprising administering to a mammal in need thereof an effective amount of one or more DHA related compounds, as well as their use as an adjuvant or co-adjuvant in a method for preventing, modulating or curing in a mammal, the chronical infection with Helicobacter pylori or the symptoms associated with H. pylori infection or related diseases, and/or preventing recurrence as well as relapse of H. pylori infection or related diseases, and/or improving the clinical condition of a mammal infected with H. pylori.

The present invention relates to a method for the preventive or curative treatment of Helicobacter pylori infections and/or related diseases.

Helicobacter pylori (HP) infection is recognized as a major etiological factor in chronic active gastritis, peptic ulcer and gastric cancer. Treatment of H. pylori infection has not significantly changed over the last decade, and drug-resistant strains of H. pylori and non-compliance to therapy are the major causes of treatment failure.

### RELATED ART

Helicobacter pylori infection is extremely common world-wide: more than one half of the world population is infected with this organism. These gram-negative bacteria are recognized as a major etiological factor in chronic active gastritis, gastric ulcers and gastric cancer, and successful treatment of this pathogen often leads to regression of some of its associated diseases¹. The outcome of the infection depends on complex interactions established between the bacteria and the host, such as the virulence of the infecting strain, the host genetic background, and the environmental factors².

Treatment of the H. pylori infection has not changed in the last decade. The treatment regimen recommended at several consensus⁴⁴⁻⁴⁸ for worldwide use is a triple therapy consisting of a proton pump inhibitor, and two antibiotics such as amoxicillin and clarithromycin given for a week.

However, treatment failures are observed due to a high incidence of antibiotic resistance. Prospective multicentre surveys have been carried out in Europe, and an important difference is noted between the Northern and Southern parts of Europe concerning clarithromycin resistance: for adults in the Northern Europe the global prevalence is less than 5%, while in Southern Europe it is as high as up to 20% or more³⁻¹⁵. Outside Europe the prevalence of clarithromycin resistance tends to be lower. Nevertheless, it has already reached 10-15% in the USA based on data from clinical trials¹⁶⁻¹⁸. The essential risk factor for clarithromycin resistance is previous consumption of macrolides. The incidence of the resistance is higher in children during the last decade, because of the increased prescription of these drugs, for respiratory tract infections¹⁹.

Other treatments have also been proposed including metronidazole, a drug for which resistance is also a problem although to a lesser extent. In contrast to clarithromycin, metronidazole resistance does not change significantly between Northern and Southern Europe countries. The global resistance rate to metronidazole is of 33.1% (95% Cl 7.5-58.9), but with a significantly lower prevalence in Central and Eastern parts (29.2% (95% Cl 17.9-41.5)) (p<0.01)²⁰. The most important risk factors in metronidazole resistance are the past use of this antibiotic for parasitic diseases in tropical countries, and in gynecological infections in women²⁰.

Resistance to amoxicillin, tetracycline, rifabutin and also fluoroquinolones is for now very low or even absent in most countries. However, in Portugal the rate of resistance to fluoroquinolones can achieve 20.9% as it has been described by Cabrita et al, in strains isolated from 110 adult patients, reflecting the misuse of these antibiotics¹¹. Discontentedness with H. pylori eradication regimen is therefore growing in most developed countries²¹. Rapid emergence of antibiotic resistance, as pointed out before, possible recurrence of infection, high cost, side-effects and poor compliance of pharmacological therapy are increasing the need for an effective new therapeutic strategy against H. pylori.

Two major strategies have been developed to overcome the failure of H. pylori eradication therapies, namely, the generation of a vaccine to stimulate the host immune defenses, and the development of new and more potent substances that could inhibit bacterial growth²².

Although extensive studies in H. pylori mouse model have demonstrated the feasibility of both therapeutic and prophylactic immunizations, the mechanism of vaccine-induced protection is still poorly understood, meaning that vaccination as a tool to eradicate H. pylori is still very controversial²³.

Various nutrients²⁴⁻²⁶ and fatty acids²⁶ have been described to exhibit an inhibitory effect on bacterial growth. Interest for the role of polyunsaturated fatty acids (PUFAs) was stimulated by Hollander and Tarnawski, who have linked the decline in duodenal ulcer with the rise in dietary intake of PUFAs²⁷. Moreover, it has been demonstrated that concentrations of 5×10⁻⁴ M of Linolenic acid (LA) could inhibit the growth of H. pylori in vitro²⁸.

A mechanism for PUFAs protective and inhibitory action has been proposed, involving their ability to modulate the synthesis of mucosal anti-inflammatory prostaglandins²⁷, such as Prostaglandin E₂.

The inventors have shown that DHA's antibacterial effect, unlike demonstrated by other reports²⁹, seems to be independent from lipid peroxidation as it is observed in an anaerobic environment.

Despite the anti-microbial effects of fatty acids on the growth of fungi, protozoan, viruses and numerous types of bacteria³⁰⁻³⁴ being well documented, only few studies have described their effects on H. pylori growth and viability²⁷, and none are available on the effect of Docosahexaenoic Acid (DHA) both in vitro and in vivo.

US2007/0021508 of Yen et al.³⁷ discloses that certain short-chain fatty acids have an ability to inhibit the growth of H. pylori, in particular, 2-phenylbutyrate and 4-phenylbutyrate. The short-chain fatty acids have the formula R₁R₂R₃C—(CH₂)ₙ—C(O)—OH wherein R₃ is aryl or heteroaryl, n can be 6 and R₁ and R₂ are H or a C₁-C₈-alkyl chain. However, not all other phenylbutyrate-like compounds have similar anti-H. pylori activity.

US2003/0032674 of Hwang³⁸ discloses the use of unsaturated fatty acids that are essentially free of saturated fatty acids for ameliorating or preventing the symptoms of a severe inflammatory disorder associated with activation of a Toll-like receptor. Various in vitro assays have been conducted to determine or suggest that DHA C22:6n-3 and EPA C20:5n-3 could be useful as inhibitors in certain activation pathway of COX-2 and therefore would be useful against severe inflammatory diseases. H. Pylori related diseases are not suggested, neither any effect of fatty acids on chronical inflammatory diseases.

For some bacterial species it has been demonstrated that certain lipids have a growth inhibitory effect. Thompson et al.²⁸ have shown that incubation of H. pylori with certain fatty acids induces an inhibitory effect on H. pylori growth, after exposure to concentrations of 0.5 mM and to 1 mM. More precisely, according to the in vitro assay conducted by Thompson et al., concentrations of 0.2 mM of linolenic acid ω-3C18:3 and ω-6C18:3 as well as EPA (C20:5) could inhibit the growth of H. Pylori, compared to oleic C18:1, linoleic C18:2 or arachidonic C20:4 acid.

Drago et al⁴¹ conducted in vitro studies concerning three formulations of fish oils comprising PUFAs and their effect on the growth of H. pylori at various oil concentrations. The effect was shown to be dependant on the formulation and on the ratio between the PUFAs. There is no information on the putative effect of each of the fatty acids per se.

Frieri et al⁴² described PUFA supplementation using fish oil and blackcurrant seed oil together with Vitamin E (antioxidant). The fish oil comprises a mixture of at least seven PUFAs without precise indication of the potential effect of each of the component.

JP10-130161³⁹ discloses the antibacterial action against H pylori of α-linolenic acid in combination with liquorice oil extract. The described pharmaceutical compositions comprise a compound having an antibacterial effect (for example glycyrrhiza) as well as at least two free long chain fatty acids, including DHA, which is associated with at least one other PUFA such as EPA.

There is still a need for a method for preventing or curing or H. pylori infections and related diseases, particularly diseases related to chronical H. pylori infections, such as gastritis, peptic ulcer and gastric cancer, or for improving the condition of a patient infected by H. pylori or affected by such a related disease, that would avoid at least the disadvantages of resistance to usual antibiotics and/or of high treatment costs.

There is still a need for effective and less toxic treatments to eradicate H. pylori infections and to prevent the occurrence or the development of the diseases related to H. pylori infection, particularly related to H. pylori chronical infections.

The present invention relates to preventing, modulating or curing in a mammal, the infections with Helicobacter pylori or the symptoms associated with H. pylori infection(s) or related diseases, and/or improving the clinical condition of a mammal infected with H. pylori, or affected by a related disease, comprising administering to a mammal in need thereof an effective amount of one or more DHA related compound(s) selected from the group comprising the docosahexaenoic acid (DHA), pharmaceutically acceptable salts thereof, esters or derivatives thereof, as well as pharmaceutically acceptable precursors or prodrugs thereof and metabolites thereof and their mixtures.

As it appears in the examples below, the inventors performed an in vitro dose-response study of H. pylori growth in the presence of docosahexaenoic acid (DHA), an essential n-3 polyunsaturated fatty acid (PUFA), present especially in fish oil. DHA has been shown to have the ability to decrease growth of H. pylori in vitro and also inhibit/reduce colonization in mice gastric mucosa in an in vivo model.

Thompson et al, in 1994, have shown that incubation of H. pylori with certain fatty acids induces an inhibitory effect on H. pylori growth, after exposure to concentrations of 0.5 mM and to 1 mM.

The inventors however demonstrated that DHA decreases H. pylori growth in a concentration dependent manner.

Furthermore, DHA addition after 12 hours of H. pylori liquid culture induces a significant decrease in a dose dependent manner of the number of CFU of all strains of H. pylori tested, suggesting a bactericidal effect of DHA on H. pylori growth. Whereas up to 100 μM DHA seems to have a bacteriostatic effect, thereby slowing or stationing the rate of growth of H. pylori, it became bactericidal to H. pylori at higher concentrations. In other words, concentrations of DHA higher than 100 μM completely arrest H. pylori growth illustrating a bactericidal effect, while concentrations of DHA lower than 100 μM decrease H. pylori growth rate, showing a bacteriostatic effect. These results were obtained in vitro. Both bactericidal and bacteriostatic effects were obtained with doses 5 to 10 times lower than the prior art doses.

Furthermore, the activity of DHA in the bacterial cultures results in alterations of bacterial cell surface as observed by electronic microscopy.

The action of DHA against H. pylori has also been investigated in vivo.

The effect of DHA on H. pylori was studied in different ways, as it appears in the examples below as illustrated by the figures.

1—The role of DHA on mice gastric colonization has been assessed with various periods of infection. Infected mice have started on DHA treatment for the whole period of the experiment by oral route, then have been sacrificed after various months of infection. An inhibition of H. pylori growth of approximately 10-fold has been observed within the one, three and six month's time-point; on the nine months of infection the inhibition effect of DHA was even higher, of about 100-fold.

2—The DHA effect has also been evaluated in mice gastric colonization when given prior to the infection. Therefore, mice have been supplemented with DHA, for three months, then infected with H. pylori strain SS1 for three, six and nine months. The treatment with DHA has been continued over the whole experiment. The inhibition of mice gastric colonization was higher within longer periods of infection (6 and 9 months).

3—The efficacy of DHA versus standard therapy has been compared in H. pylori eradication: Mice have been infected with H. pylori SS1 strain for one month and treated with DHA for 15 days or/and standard therapy for 7 days, as described elsewhere (Lee et al. (52)). When DHA has been administered as an adjuvant to the standard therapy, the efficacy in the inhibition of gastric colonization has been 10 times stronger. Furthermore, DHA-treated mice presented a lower H. pylori gastric colonization (10-fold).

4—H. pylori infection is associated with a chronic inflammation of the gastric mucosa. In order to assess the mice inflammation status and its relation with DHA supplementation, the serum prostaglandin E2 (PGE₂) levels have been analyzed and it has appeared that the consumption of DHA had a drastic inhibitory effect in serum PGE₂ levels. The inflammation of the gastric mucosa being more severe in the antrum as compared to fundus, independently of the time-point of infection, the infected-mice supplemented with DHA have presented lower inflammation scores when compared to infected non-supplemented ones, either after 6 or 9 months. Moreover, when mice have been treated with DHA for 3 months prior to the infection, they have shown even a lower inflammation score.

5—The action of DHA on the ability of the H. pylori infection to induce an inflammatory effect associated with an induction of gastric neoplasic lesions has also been investigated in vivo in the INS-GAS mouse model, which mice are transgenic for the human gastrin and develop spontaneously gastric cancer lesions, exacerbated in the presence of the H. pylori infection (52; 43). By comparison between mice, which have been orogastrically infected with the H. pylori strain SS1 as previously described⁴⁹ and non-infected mice, a half of the animals having been treated with DHA, in their drinking water has shown an inhibitory effect on the H. pylori infection in vivo as observed by the decrease of the H. pylori antigen-specific antibody response. A lower level of PGE₂ in the sera has also been observed, relating to an anti-inflammatory action of DHA.

6—As previously reported in the mouse model of infection (Touati 49), the analysis of the gastric inflammatory lesions of the infected mice, has shown the presence of infiltrates of PMN and plasmocytes in the infected mucosa with score grading slightly decreased in the presence of DHA only observed in the fundus.

Furthermore, an histopathological analysis and grading of gastric lesions has been performed, as illustrated in the figures. Concerning to histological lesions, the presence of DHA decreases the severity of hyperplasic lesions with less architectural atypies that were observed in SS1-INS-GAS infected mice.

In conclusion, the results demonstrate that DHA inhibits H. pylori growth in a dose-dependent manner, both in vitro and in vivo.

These data observations pave the way for the use of DHA in preventive and curative strategies for H. pylori infection, or as an adjuvant agent or co-adjuvant agent in H. pylori eradication and/or to prevent recurrence as well as relapse of H. pylori-infection, and H. pylori-associated or -related disease(s).

Particularly, the settlement or implantation of H. pylori or the colonization by H. pylori of the mucosa and of the stomach as the consequence can be prevented or at least can be controlled.

In contrast to the evident efforts to demonstrate H. pylori implication in gastric cancer, little had been done to date regarding the putative dietary influences on its growth and survival. As mentioned above, although H2 blockers, proton-pump inhibitors and antibiotics are effective, relapses are not only still occurring, as well as they are quite common²¹.

It should be emphasized that fatty acids used according to the invention are not only much less toxic than standard therapeutic agents, but also well tolerable, and therefore could be given for long periods of time, i.e. in a long-term therapy regimen.

As a consequence, DHA supplementation in at risk group diets as a reasonable and safe prophylactic/preventive strategy can be considered.

More details and advantages will be apparent in the following detailed description and examples as illustrated by the figures. On the figures, “−” denotes the colonization or the grading average.

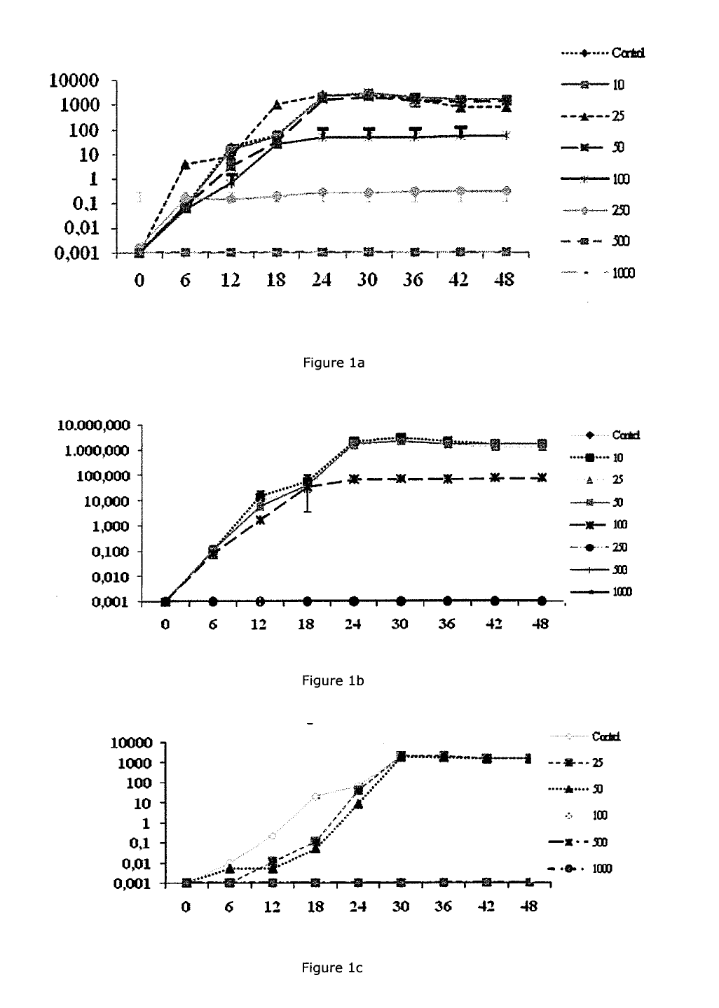

FIG. 1a illustrates the in vitro growth inhibition of H. pylori by DHA addition (for various DHA μM concentrations) at the time 0, on strain 26695 in log 10⁷ in CFU. H. pylori was evaluated every 6 hours.

FIG. 1b illustrates the in vitro growth inhibition of H. pylori by DHA addition (for various DHA μM concentrations) at the time 0, on strain SS1 in log 10⁷ in CFU. H. pylori was evaluated every 6 hours.

FIG. 1c illustrates the in vitro growth inhibition of H. pylori by DHA addition (for various DHA μM concentrations) at the time 0, on strain B128 in log 10⁷ in CFU. H. pylori was evaluated every 6 hours.

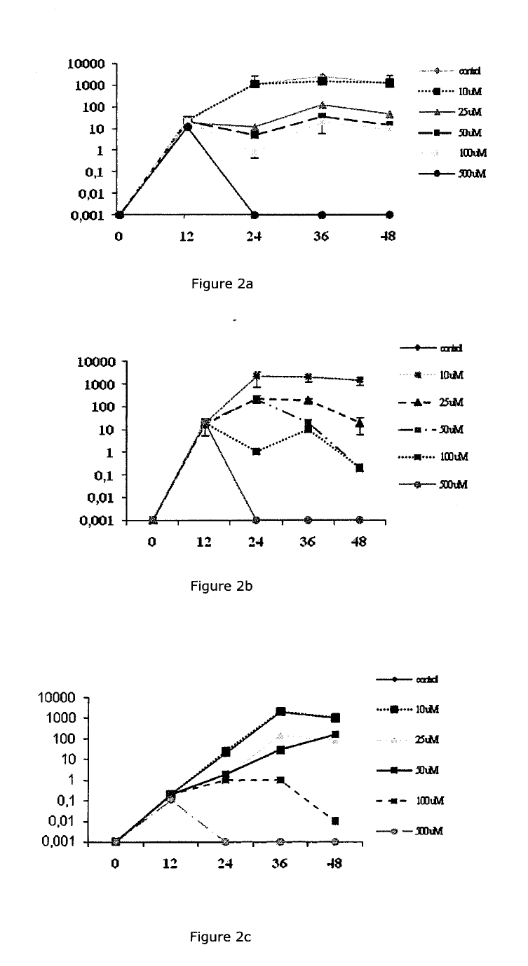

FIG. 2a illustrates the in vitro growth inhibition of H. pylori by DHA addition (for various DHA μM concentrations) after 12 hours, on strain 26695 in log 10⁷ in CFU. H. pylori was evaluated every 12 hours.

FIG. 2b illustrates the in vitro growth inhibition of H. pylori by DHA addition (for various DHA μM concentrations) after 12 hours, on strain SS1 in log 10⁷ in CFU. H. pylori was evaluated every 12 hours.

FIG. 2c illustrates the in vitro growth inhibition of H. pylori by DHA addition (for various DHA μM concentrations) after 12 hours, on strain 8128 in log 10⁷ in CFU. H. pylori was evaluated every 12 hours.

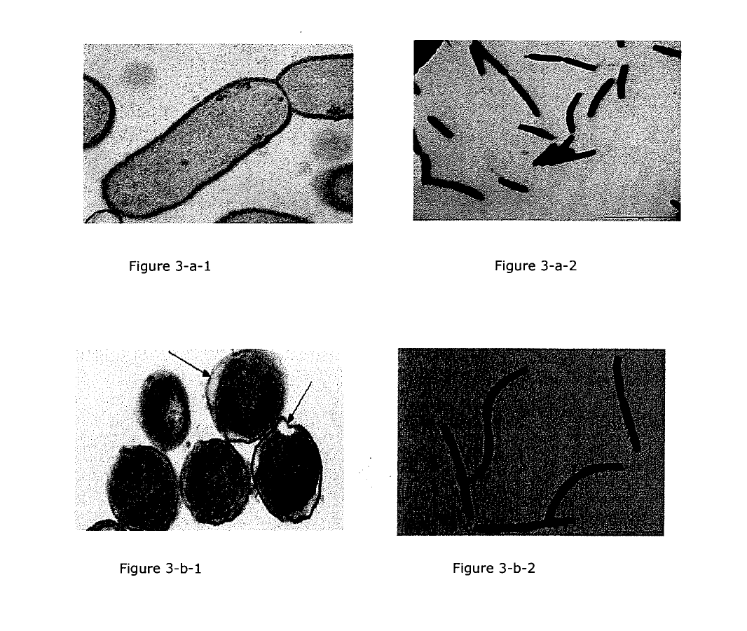

FIG. 3a illustrates the morphology of H. pylori strain 26695 in the absence of DHA compared with the morphology of H. pylori strain 26695 treated with DHA (100 μM) in liquid cultures for 12 hours illustrated in FIG. 3b.

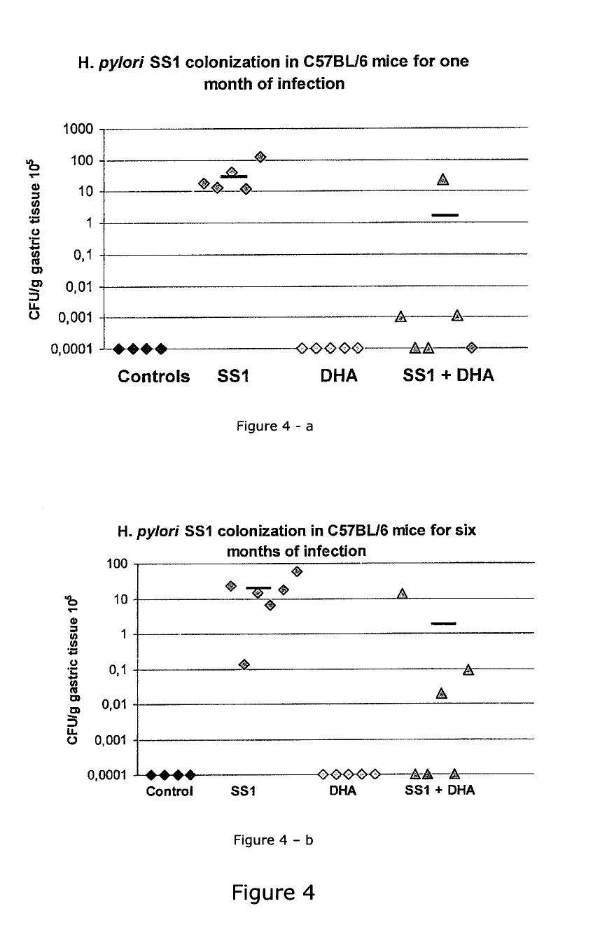

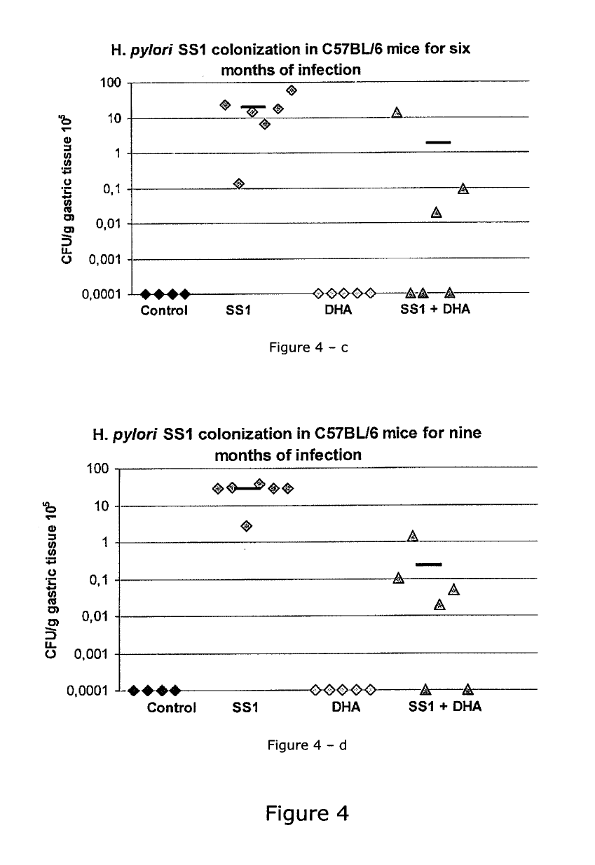

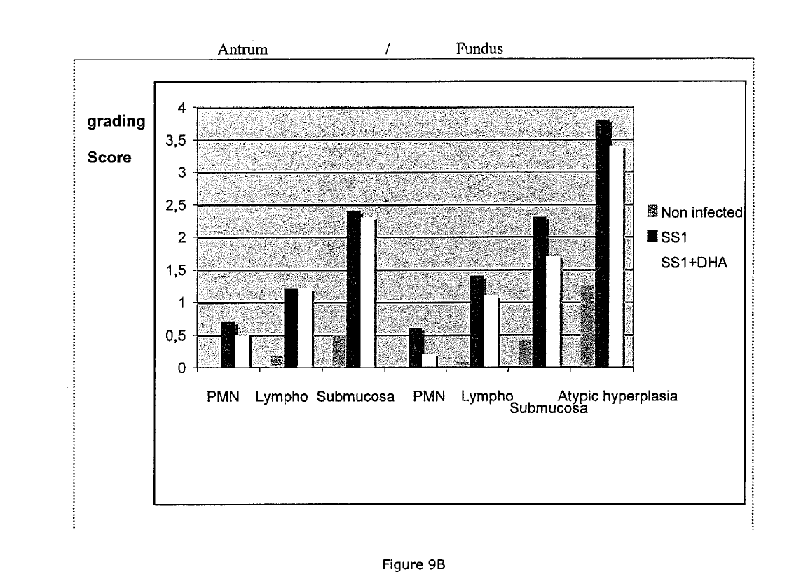

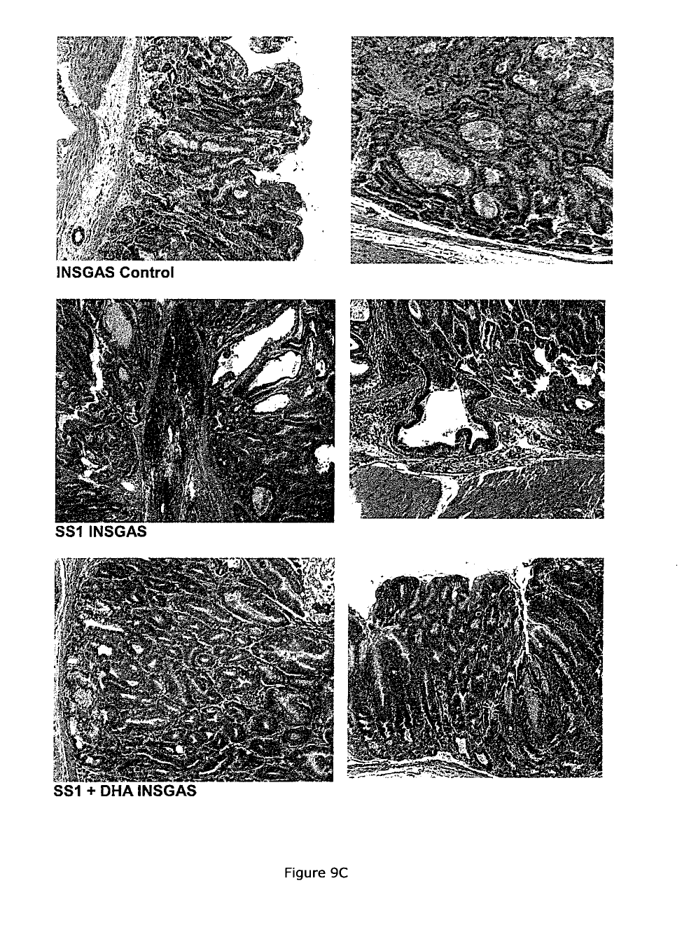

FIG. 4a to FIG. 4d illustrates the colonization of C57BU6 mice gastric mucosa for 1 month (FIG. 4a), 3 months (FIG. 4b), 6 months (FIGS. 4c) and 9 months (FIG. 4d) of H. pylori infection with strain SS1 with and without DHA 50 μM in the drinking water.

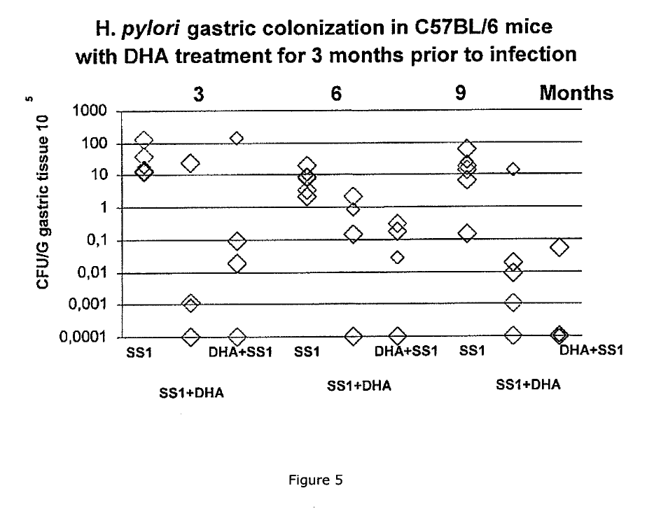

FIG. 5 illustrates the colonization of C57BU6 mice gastric mucosa with DHA given for 3 months prior to infection for 3, 6 and 9 months with strain SS1 (DHA+SS1), versus C57BU6 without DHA prior to infection (SS1+DHA), DHA being administered during the all experiment.

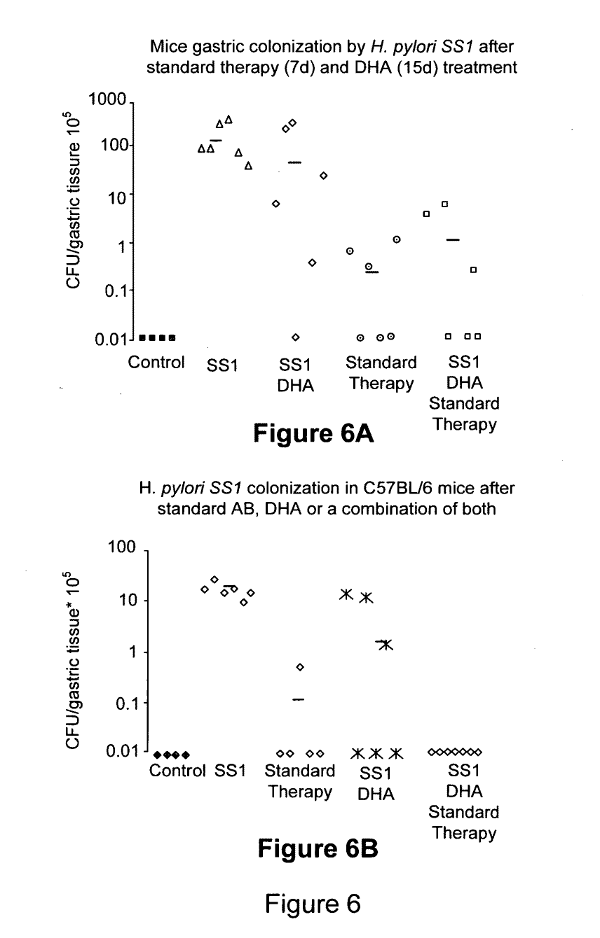

FIG. 6A illustrates the colonization of C57BU6 mice gastric mucosa after DHA treatment (SS1 DHA); standard therapy; and standard therapy in combination of DHA treatment (SS1 DHA Standard therapy). FIG. 6B illustrates the colonization of C57BU6 mice gastric mucosa after DHA treatment (SS1 DHA); standard therapy AB and standard therapy in combination of DHA treatment (SS1 DHA Standard therapy). AB relates to AntiBiotics.

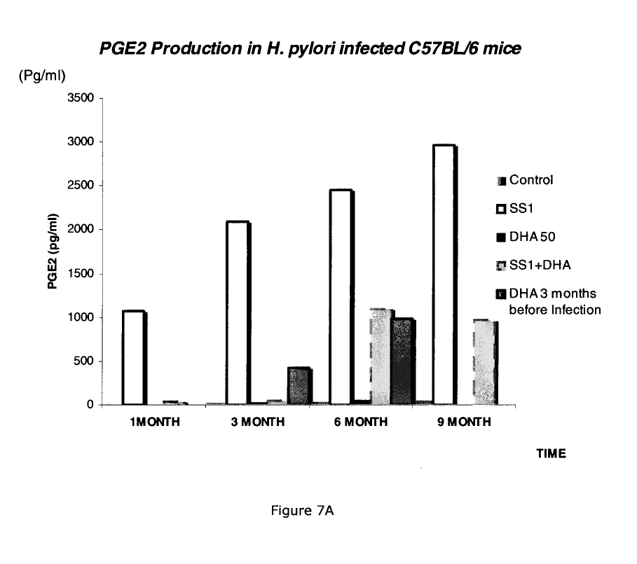

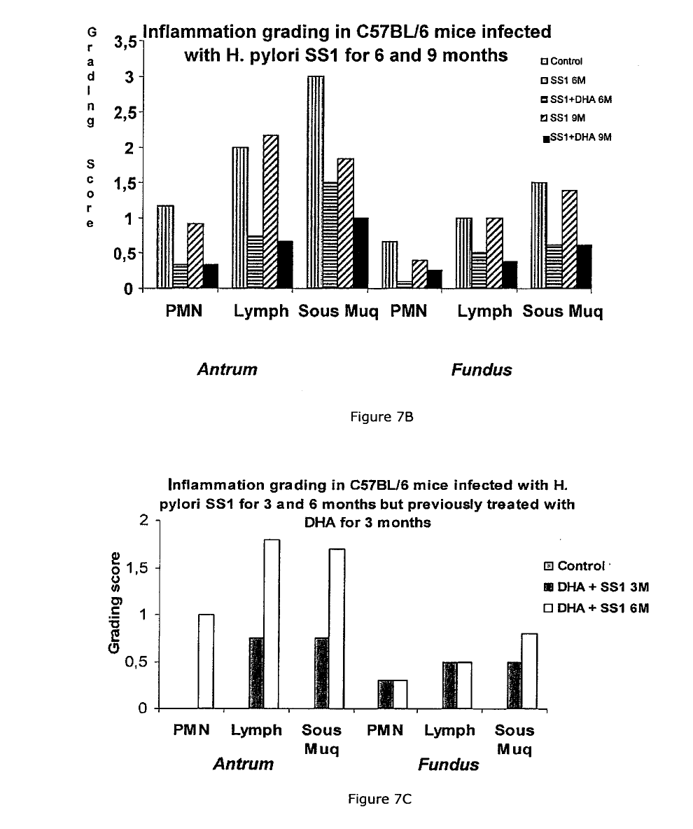

FIG. 7A illustrates the production of PGE2 (pg/ml) in the sera of C57BU6 mice infected for 1, 3, 6 and 9 months with H. pylori strain SS1, with and without DHA 50 μM in the drinking water.

FIG. 7B illustrates the gastric inflammation scores grading of C57BU6 mice infected for 1, 3, 6 and 9 months with H. pylori strain SS1, with and without DHA 50 μM in the drinking water.

FIG. 7C illustrates the gastric inflammation scores grading of C57BU6 mice gastric mucosa with DHA given for 3 months prior to infection for 3 and 6 months with strain SS1, DHA having been administrated during the all experiment.

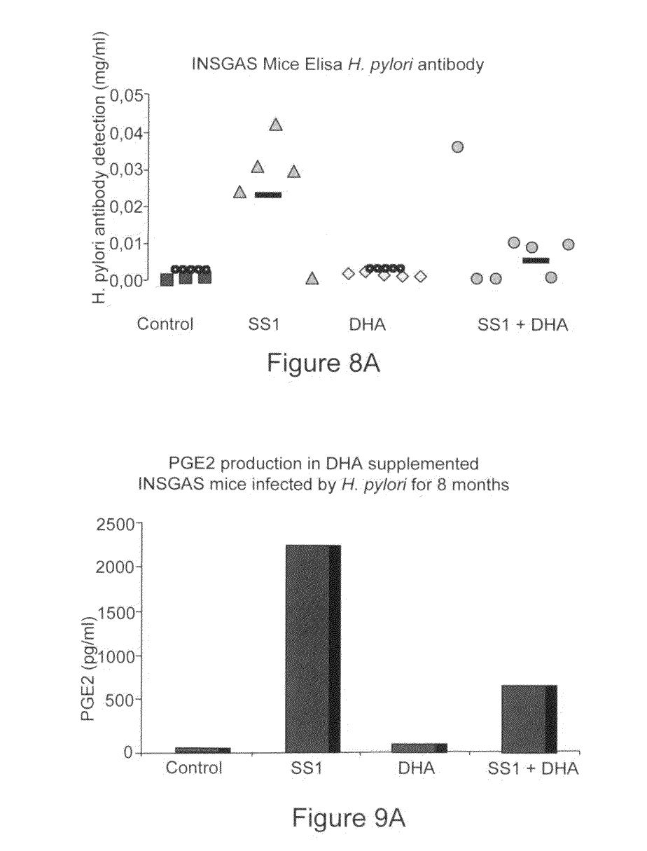

FIG. 8 illustrates the H. pylori antibodies production of INS-GAS mice gastric mucosa in the FVB genetic context, for 8 months of H. pylori infection with strain SS1 with and without DHA 50 μM in the drinking water.

FIG. 9A illustrates the production of PGE2 in the sera of INS-GAS mice gastric mucosa in the FVB genetic context, infected for 8 months with H. pylori strain SS1 with and without DHA 50 μM in the drinking water.

FIG. 9B illustrates the gastric inflammation scores grading of INS-GAS mice gastric mucosa in the FVB genetic context, infected for 8 months with H. pylori strain SS1 with and without DHA 50 μM in the drinking water.

FIG. 9C illustrates an histopathological analysis, i.e. the histological lesions observed in the gastric mucosa of the INS-GAS mice, infected for 8 months with H. pylori strain SS1 with (SS1+DHA INS-GAS) and without (SS1 INS-GAS) DHA 50 μM in the drinking water.

The term treatment as used in the present application refers to administering a composition to a mammal, preferably to a human patient, with the purpose of preventing, modulating, or curing H. pylori infection(s) or symptoms associated with H. pylori infection(s) or a disease related to H. pylori infection, including chronic infection, as well as the prevention of recurrent infections, or for improving the clinical condition of a mammal, especially a human patient who has been infected with H. pylori, or is affected by at least one related disease.

Among H. pylori associated or related diseases, the present invention relates to chronic atrophic gastritis which is a precancerous lesion for gastric cancer. As the eradication of H. pylori can stop or ameliorate chronic gastritis, eradication of H. pylori may prevent gastric cancer. The present invention also relates to stopping, inhibiting or decreasing lesion of atrophic gastritis until the stage just preceding dysplasia.

According to the present invention, related disease or associated disease includes active gastritis, chronic atrophic gastritis, gastric ulcer, duodenal ulcer, peptic ulcer disease, gastric cancer, peptic esophagitis, gastric adenocarcinoma, mucosa-associated lymphoid tissue lymphomas and the like.

In a particular embodiment of the present invention, the administration of the DHA related compound(s) prevents, decreases, alleviates, or abolishes chronic inflammation resulting from H. pylori infection(s) in a mammal.

DHA is a long chain fatty acids of the n-3 series (ω-3). DHA (22:6n-3) may be used in its free acid form, or in the form of pharmaceutically acceptable forms. DHA is an essential PUFA present mainly in fish and marine oils, from which it can be extracted to prepare the compounds of the invention.

According to the present invention, pharmaceutically acceptable forms of DHA are salts, esters or derivatives, precursors or prodrugs, or metabolites of DHA, which, as well as DHA in its free acidic form, are herein “DHA-related compound”, i.e.; generally any substance or compound which is pharmaceutically acceptable and able to deliver DHA in a biologically active form in the stomach lumen. DHA metabolites are herein included in DHA-related compounds, in so far as said metabolites according to the inventor are biologically active forms of DHA.

By “biologically active form” of the compounds, it is herein intended any form, which can prevent, treat, cure, modulate or improve conditions linked with the presence of H. pylori or related diseases or symptoms associated herewith.

Derivatives of DHA are chemical compounds obtainable from DHA by usual chemical reactions, which do not affect the biological activity thereof.

Examples of pharmaceutically acceptable salts include alkali metal salts such as potassium or sodium salts, alkaline metal salts such as calcium salt or magnesium salt, ammonium salts, salt with a organic base such as triethylamine salt or ethanolamine salt.

Examples of derivatives are esters.

Examples of esters are DHA C₁-C₂₂ alkyl esters and their mixtures, preferably C₂-C₂₂ alkyl esters and their mixtures, more preferably, C₂-C₈ alkyl esters and their mixture. Amongst them, DHA ethyl ester is preferred.

Other examples of esters include glycerin esters. The glyceride include for example monoglyceride, diglyceride, triglyceride, as well as medium-chain glyceride as well as structured triglycerides.

According to the present invention, prodrugs or precursors of DHA are compounds and substances which are able to provide DHA, in one of its biologically active form, preferably in its free acidic form, when delivered to an individual.

Examples of such precursors are di- or triacyl-glycerol fatty acids, which can be released as DHA in its free acidic form. Further examples include phospholipids in which phosphatidic acid in which two fatty acids are esterified to an hydroxyl group of glycerin and a phosphoric acid is bound to the third hydroxyl group is a basic skeleton and choline, ethanolamine, serine, inosine or the like is phosphodiester-linked to the basic skeleton. Examples of the phospholipids include lecithin, kephalin, phosphatidylserine, sphingolipid and sphingomyelins.

Metabolites are in particular oxygenated metabolites derived from DHA. These metabolites are known as resolvins of the D series when derived from DHA. Examples of such resolvins are given below.

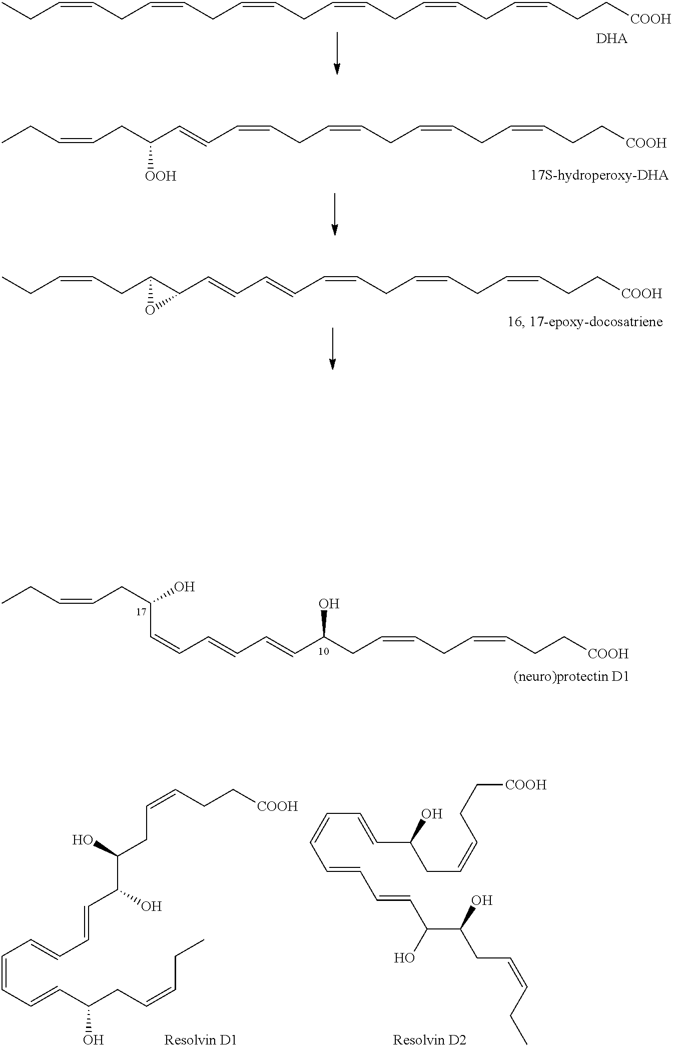

Some of these DHA related compounds could need to be associated with another entity to be able to release DHA in one of its biologically active form in the stomach lumen, and, amongst them, DHA in its free acidic form in the stomach lumen.

Preferably according to the invention, the DHA is used in its free acidic form.

An effective amount of a DHA-related compound is the amount which, upon administration to an individual in need of treatment of H. pylori infection or of at least a disease associated or related to H. pylori, is required to confer an effect on said individual.

As used herein, an “amount of DHA” is the amount of its free acidic form whatever the biologically available form as delivered in the stomach lumen.

In a further embodiment of the present invention, DHA or its related compounds or mixtures thereof are (is) used alone, i.e. without any other PUFA's.

Based on the available evidence, usual dosage in different applications range between 0.1-10 g/day, preferably 0.5-6 g/day, more preferably 1 g/day. The dosage varies between 0.5 g and 1.0 g for human.

In a preferred aspect of the invention, the dosage may range from 1 mg/kg/day to 10 mg/kg/day of DHA, more preferably 1.8 mg/kg/day to 6 mg/kg/day of DHA.

For a man of an average body weight of 70 kg, the daily dose of DHA is comprised between 100 mg and 500 mg, preferably 120 mg and 420 mg.

In a further embodiment of the invention, the therapeutically active dosage prevents H. pylori to settle or implant in the mucosa, with the same doses as above mentioned.

DHA was available in mice drinking water in a concentration of 50 μM, which corresponds to 0.821 mg (molar mass of DHA is 328.48 g/mol), and was changed every two days. In average, cages are shared by six mice, meaning that each mice received every day 0.068 mg of DHA. According to references the amount of DHA given to every mouse is about ten times higher, which became important to warranty that even in such high doses there seems to be no toxicity concerns.

It is important to mention that several doses were tested (25, 50 and 100 μM) and that 50 μM was the one that showed a higher inhibitory role in gastric colonization.

The dosage preferably ranges for human from 1.8 to 6 mg/kg/day.

However, according to an embodiment, the concentration of DHA solution in the stomach lumen is between 25 mM and 100 mM, preferably between 40 mM and 60 mM.

According to a particular embodiment, interestingly, the concentration of the DHA solution has to be around 50 μM in the stomach lumen.

The DHA doses for administration are between 100 and 800 mg/day, preferably 200 to 500 mg/day. Examples of daily dosages are of 450 mg/day +/−10%, 500 mg/day +/−10%, 610 mg/day +/−10% and 667 mg/day +/−10%.

For a man of about 70kg, the daily dosage per kg is between 0.0020 g/day/kg and 0.0100 g/day/kg, preferably 0.0028 g/day/kg and 0.0064 g/day/kg. Examples of such daily dosage per kg are 0.0064 g/day/kg +/−5%, 0.0071 g/day/kg +/−5%, 0.0087 g/day/kg +/−5% and 0.0095 g/day/kg +/−5%.

The effective dosis may vary depending on the route of administration, on excipient and on the biological availability of the DHA-related compound, as well as the presence of other active agents such as anti-ulcer or antibiotics. The effective amount may also depend on factors such as the age, gender, diet, body weight, health status, rate of excretion, timing of administration, the severity and stage of H. pylori infection or the related and associated diseases and on the individual disposition to the diseases and response to the treatment.

The medicament, e.g. in the form of a pharmaceutical composition, according to the present invention can be prepared according to known methods in the art.

The medicament or pharmaceutical composition can comprise the desired amount of DHA, DHA-related compound(s) or mixtures thereof and a pharmaceutical acceptable vehicle, e.g., carriers, excipients, adjuvants and buffers, for instance substances used in pharmaceuticals or known in the art (Remington's Pharmaceutical Sciences—Alfonso Gennaro).

A pharmaceutically acceptable carrier may include water, a solvent, a preservative, a surfactant, a combination of the pharmaceutically acceptable carriers and excipients.

For example, water, when present, can be in an amount of about 3 to 97% by weight. Other than water, the carrier can also contain solvent, particularly relatively volatile solvent such as monohydric C₁-C₃ alkanol, for example ethanol, in an amount of about 2% to 80% by weight and an emollient such as those in the form of silicone oils and esters.

The preferred route of administration is the oral route, however various alternative routes of administration can be considered, for example a route such as the parenteral route may be used.

In case of the oral administration, DHA or DHA related compounds or their mixtures can be administrated in the form of soft or hard capsules, tablets, powders, granules, pastes, syrups, solutions, W/O or O/W emulsions, microemulsions, suspensions, liposome formulations, microcapsules, nanocapsules, modified-release formulation such as extended release formulations or the like, particularly those adapted for stomach-coating medication or gastrointestinal protectants. The DHA or DHA related compounds or mixtures thereof can be formulated as a composition comprising in addition to said DHA or DHA related compounds or mixture thereof, suitable excipients such as diluents, stabilizers, solvents, surfactants, buffers, carriers, preservatives, and adjuvants.

Capsules may contain any standard pharmaceutically acceptable materials such as gelatin or cellulose. Tablets may be formulated in accordance with conventional procedures by compressing DHA, DHA related compounds or mixtures thereof with a solid carrier and a lubricant. Examples of solid carriers include starch and sugar bentonite. The selection of the method for the delivery of the DHA, DHA related compound(s) or mixtures thereof and their adapted vehicules, desintegrators or suspending agents can be readily made and adapted by persons skilled in the art.

The DHA or DHA related compounds of mixtures thereof according to the invention can be associated with other active compounds such as gastro-intestinal protectants, enzymatic inhibitors, such as pancreatic trypsin inhibitors, diisopropylfluorophosphate (DFF) or trasyfol antibiotics, H2 blockers, proton pump inhibitors, anti-inflammatory substances, anti-tumor compounds. Examples of said other active compounds are amoxicillin, clarithromycin, tetracycline, rifabutin, fluoroquinolones, metronidazole, a proton-pump inhibitor such as omeprazole.

According to one aspect of the invention, the DHA or DHA related compounds or mixtures thereof used according to the invention can also be used as an adjuvant or a co-adjuvant. Accordingly, DHA, DHA related compound(s) or mixtures thereof are used as an adjuvant or co-adjuvant of one or more pharmaceutically active compound in a method of preventing, modulating or curing in a mammal, the infection with Helicobacter pylori or the symptoms associated with H. pylori infection or associated or related diseases, and/or improving the clinical condition of a mammal infected with H. pylori, comprising administering simultaneously or sequentially to a mammal in need thereof an effective amount of said active compound and of said adjuvant or co-adjuvant. Examples of co-adjuvant are resorcinol, non ionic surfactants.

In view of its non-toxic properties, DHA or DHA related compound(s) or mixtures thereof may be used in treatment regimens involving administration of the active compound(s) over several weeks or months or even over one or several years, i.e. may be suitable for long-term therapy which may be necessary for patients suffering from H. pylori infection.

The DHA-related compounds according to the invention should have a similar inhibitory effect on the growth of different H. pylori strains, whatever the resistant phenotype to the various antibiotics commonly used.

More generally, the invention provides DHA, DHA related compounds or their mixtures which may be used in the formulations or in the methods of the invention for single multiple or continuous administration.

In addition, when appropriate, the DHA, DHA related compounds or their mixtures may be used for co-administration or for combination treatment with other molecules active against H. pylori infection or related disease(s). Said other active compounds may be administered simultaneously or sequentially with the DHA, DHA related compounds or mixtures thereof or may be administered at a different time during the treatment.

### EXAMPLES

### Example 1

### Materials and Methods

1. Fatty Acids and Strains

Fatty acids: DHA was purchased from Cayman Chemical Company (Michigan, USA) with a purity of approximately 98% within an ethanol mixture.

Bacterial Strain and H. pylori Culture Conditions

In this study the H. pylori strains 26695 (ATCC 700392), SS1 and B128 were used. H. pylori Strain 26695 (ATCC 700392) was obtained from the American Type Culture Collection (ATCC, Rockville, Md.), SS1³⁵ was obtained from A. Lee (Australia) and B128³⁶ was obtained from R. Peek (USA).

Bacteria were routinely cultured on Blood agar plates with the usual antibiotics and fungicide mixtures⁵¹ and incubated at 37° C. under microaerophilic conditions (GENbox anaerobic, BioMérieux, France) for 48 hours. H. pylori cultures were characterized by colony morphology and biochemical tests (urease, catalase and oxidase assays). For the preparation of bacterial broth cultures, colonies growing on agar plates were resuspended in 1 ml of Brucella broth (BB) supplemented with 10% decomplemented foetal bovine serum (FBS). Broth cultures were incubated under microaerophilic conditions as described above.

2. In Vitro Incubation of Bacterial Cultures with DHA

Stock solutions of DHA were diluted at desired concentrations (from 10 μM to 1000 μM) in Brucella broth (BB) enriched with 10% FBS. To establish H. pylori growth curves overnight bacteria cultures were diluted 100-fold in 10 ml of medium with or without DHA to an initial optical density (OD600 nm) of 0.03.

Each experiment consisting of a control (bacterial medium) and bacteria incubated with DHA at 10 μM, 25 μM, 50 μM, 100 μM, 250 μM, 500 μM and 1000 μM was performed in triplicate. DHA was added at the time 0 of the experiments or after 12 hours. H. pylori broth cultures were incubated microaerophilically, at 37° C. for 48 hours. Every 6 hours, the OD 600 nm of the cultures was measured and 200 μL samples were diluted and plated on blood agar plates for the numeration of viable bacteria (colony-forming-units (CFU)) after 48 hours of incubation at 37° C.

3: Electron Microscopy

In order to examine putative differences in morphology and structure of H. pylori induced by DHA treatment, H. pylori strain 26695 was grown for 12 hours in the presence of DHA, as described in 2 above. Following the 12 hours aliquots of H. pylori treated with DHA were withdrawn and the morphology of bacteria was observed on electron microscopy and compared with a control culture in absence of DHA. Transmission electron microscopy at ambiant temperature was carried out with Geol 1200 Ex 2-Tokyo.

4: In Vivo Assay on C57BU6

Animal C57BU6

Five-weeks-old specific pathogen-free C57BU6 male mice have been purchased from Charles River Laboratories (France). Animals were housed in microisolators in polycarbonate cages. Food has been supplied ad libitum. Standard diet was purchased from SAFE (Epinay/Orge, France). Animals were acclimatized for one week before inoculation. The experiments reported in the study were approved in advanced by the Central Animal Facility Committee of Institut Pasteur, in conformity with the French Ministry of Agriculture Guidelines for Animal Care.

Mouse Model for H. Pylori Colonization

A—

H. pylori strain SS1 was grown on blood agar plates, harvested after 24 h and suspended in peptone broth. Twenty four mice (n=24) were orogastrically inoculated 100 μL of a suspension of 10⁸ CFU/mL of H. pylori strain SS1, whereas non-infected groups of mice (n=24) were given 100 μL of peptone broth. Mice were sacrificed 1 or 3 months after infection. For each time point, the experiments consisted in 4 groups of mice, 2 groups received drinking water and for the two other groups, drinking water was supplemented with DHA at a final concentration of 50 μM. Dose of 50 μM corresponds more precisely to a quantity of 0.068 mg of DHA received by each mouse each day. Each two groups consisted of one uninfected-group (n=6) and one H. pylori strain SS1 infected-group (n=6). Stomachs were isolated from each mouse and used to measure H. pylori colonization as previously described by Ferrero et al.⁵¹

B—1 Role of DHA on Mice Gastric Colonization with Different Periods of Infection: One, Three, Six and Nine Months.

C57BL/6 mice were therefore infected with SS1 strain and start on DHA treatment for the whole period of the experiment, which was given in their drinking water in a concentration of 50 μM. Mice were sacrificed at one, three, six or nine months of infection.

B—2 Evaluation of DHA Effect in Mice Gastric Colonization when Given Prior to the Infection.

Mice were supplemented with DHA, as previously described, for three months time, and then infected with H. pylori strain SS1 for three, six and nine months. Treatment of DHA continued over the whole experiment.

B—3 Comparison of the Efficency of DHA Versus Standard Therapy in H. Pylori Eradication.

C57BU6 mice have been infected with H. pylori SS1 strain for one month and proceed to treatment with DHA for 15 days or/and standard therapy for 7 days, as described elsewhere by Lee et al.⁵² and van Zanten et al.⁵³. According to the standard therapy, mice received omeprazole (400 μmol/kg/day), metronidazole (14.2 μmol/kg/day) and clarithromycin (7.15 μmol/kg/day)⁵³.

B—4 Mice Inflammation Status and its Relation with DHA Supplementation

Since H. pylori infection is associated with a chronic inflammation of the gastric mucosa, serum prostaglandin E2 (PGE₂) levels have been analyzed.

The intensity of the lesions has been evaluated semiquantitatively, according to Eaton et al⁵⁰.

0, no infiltrates of polymorphonuclear cells (PMN) and plasmocytes; 1, mild, multifocal infiltration; 2, mild, widespread infiltration; 3, mild, widespread, and moderate multifocal infiltration; 4, moderate, widespread infiltration; 5, moderate, widespread, and severe multifocal infiltration.

Lymphoid aggregates were graded as 1 (mild, 1-10 glands), 2 (moderate, 10-20 glands), or 3 (severe, more than 20 glands).

6: In Vivo Assay on INS-GAS

Animal INS-GAS Mouse Model

These mice are transgenic for the human gastrin and develop spontaneously gastric cancer lesions, exacerbated in the presence of the H. pylori infection. INS-GAS mice in the FVB genetic are disclosed in Wang et al⁴⁰.

A—Investigation of the Action of DHA on the Ability of the H. Pylori Infection to Induce an Inflammatory Effect Associated with an Induction of Gastric Neoplasic Lesions in the INS-GAS Mouse Model (Fox et al.⁴³ Wang et al.⁴⁰)

Fourteen male mice of 6-7 weeks-old were orogastrically infected with the H. pylori strain SS1 as previously described. Seven of these mice received a drinking water containing DHA (50 μM) for all the duration of the experiment. In parallel, a group of 12 non-infected mice with half of the animals treated with DHA as described above were also included in the study. After 8 months, mice were sacrificed and gastric tissues and blood collected.

B Histopathological Analysis and Grading of Gastric Lesions.

The analysis has been done in the same manner as in point 5 B-4 above

### Example 2

1—In Vitro Inhibition Results: DHA Inhibits H. Pylori Growth

The effects of DHA on the H. pylori growth and viability were first analysed on bacterial culture of 3 different strains of H. pylori, 26695, SS1³⁵ and B128³⁶. In the absence of DHA, bacteria grew steadily reaching a maximum viable count at 18-20 hours of approximately 5.64×10⁸ CFU/ml. Afterwards CFU formation rate became stationary. The presence of DHA in the growth medium led to an inhibition of H. pylori viability, which is dose-dependent (FIG. 1). Up to a concentration of 50 μM of DHA, the viability of the 3 H. pylori strains analyzed was not affected. Regardless the H. pylori strain tested, concentrations of DHA up to 100 μM induced a slight inhibition of H. pylori growth, while concentrations higher than 100 μM of DHA affected strongly the bacterial viability. However, it is to be noticed that the sensitivity of the 3 strains seemed different since the bacterial viability was 100 times higher at DHA 250 μM for strain 26695 as compared to SS1. In addition strain B128 needed more time to reach the exponential phase of growth as compared to 26695 and SS1. At concentrations of 500 μM or higher of DHA, no survival was observed for the three different strains analyzed, as said stains were not able to form colonies.

2—DHA Bactericidal and Bacteriostatic Effect: Results

The question as to whether DHA has a bactericidal effect was answered by conducting an experiment where DHA was added to a 12 hours H. pylori culture. FIG. 2 represents the viability of the three strains of H. pylori used. Addition of DHA after 12 hours of H. pylori growth led to a decrease of the ability to form CFU in a dose dependent manner. Viability of H. pylori strains decreased or maintained stationary when treated with concentrations of DHA lower than 100 μM, suggesting a bacteriostatic effect. When added doses of DHA were higher than 100 μM, H. pylori growth decreased rapidly, with a complete inhibition of bacterial viability, which is indicative of a bactericidal effect. The same response was observed for the three strains analyzed. These data indicated that DHA had an inhibitory effect on the growth and survival of H. pylori, in a dose dependent manner.

### Example 3

### Study of DHA Deleterious Effects by Electronic Microscopy Observation of the Alteration of H. Pylori Shape: Results.

FIG. 3 depicts differences in the morphology of H. pylori strain 26695 induced by DHA treatment. In the presence of 100 μM of DHA, H. pylori became more stretched and elongated when compared with controls. It is also noteworthy that the membrane of H. pylori treated with DHA exhibited “hole” structures (pointed out in FIG. 3 by arrows) that are absent in H. pylori controls, suggesting changes in H. pylori membrane.

### Example 4

### Results C5713116 Mice

In Vivo Assay: Inhibitory Effect of DHA on H. Pylori Gastric Colonization

A—The consequences of DHA on the H. pylori colonization of the gastric mucosa were investigated in the mouse model, by addition of this fatty acid in the drinking water of animals during all the time of the experiments as described in materials and methods. Despite the duration of infection, one, three, six and nine months, H. pylori SS1 infected mice which received DHA showed significantly less stomach colonization than infected mice which received only water, 5.13×10⁵ versus 0.47×10⁵ CFU/g gastric tissue (DHA non-supplemented and DHA supplemented) after 1 month of infection, and 2.99×10⁵ versus 0.62×10⁵ CFU/g gastric tissue (DHA non-supplemented and DHA supplemented) after the third month (p<0.01).

For the mice which received DHA, the colonization by H. pylori during the first month of infection only occurred in one mouse (20%) and on the third month in 3 mice (50%) but with a low level of colonization as compared to the infected mice that received only water, for the same time point. Thus anti-H. pylori effect of DHA is also achieved in vivo since the consumption of DHA significantly affected the H. pylori colonization in mice gastric mucosa.

B—1 Role of DHA on Mice Gastric Colonization with Different Periods of Infection: One, Three, Six and Nine Months.

FIG. 4 depicts DHA inhibitory effect in mice gastric colonization. An inhibition of H. pylori growth of approximately 10-fold was observed within the one, three and six month's time-point; on the nine months of infection the inhibition effect of DHA was even higher, of about 100-fold.

B—2 Evaluation of DHA Effect in Mice Gastric Colonization When Given Prior to the Infection.

FIG. 5 shows how administration of DHA prior to the infection impacted H. pylori gastric colonization. The inhibition of mice gastric colonization was higher within longer periods of infection (6 and 9 months).

B—3 Comparison of the Efficency of DHA Versus Standard Therapy in H. Pylori Eradication.

Standard therapy was more efficient in inhibiting gastric colonization of H. pylori when compared to DHA (100 times vs 10 times lower gastric colonization). However, when DHA was administered as an adjuvant to the standard therapy the efficacy in the inhibition of gastric colonization is 10 times stronger. Still, it is important to mention that, even less efficient than standard therapy, DHA presented a lower H. pylori gastric colonization (10-fold). Results concerning this experiment are depicted in FIG. 6.

B—4 Mice Inflammation Status and its Relation with DHA Supplementation

Serum prostaglandin E2 (PGE₂) levels have been analyzed.

As illustrated in FIG. 7A, the consumption of DHA had a drastic inhibitory effect in serum PGE₂ levels.

The intensity of the lesions has been evaluated semiquantitatively, according to Eaton et al (50), Lymphoid aggregates were graded.

The inflammation has been more severe in the antrum as compared to fundus, independently of the time-point of infection (FIG. 7B).

On FIG. 7B, as well as on FIGS. 7C and 9A, “PMN” means Polymorphonuclear cells, “Lympho” means lymphocytes, “sous muq” means submucosa.

Infected-mice supplemented with DHA have presented lower inflammation scores when compared to infected ones non-supplemented with DHA, either after 6 or 9 months. Moreover, when mice had been treated with DHA for 3 months prior to the infection, they have shown even a lower inflammation score (FIG. 7C).

### Example 5

### Results INS-GAS

As reported in FIG. 8, also in the INS-GAS genetic background presence of DHA 50 μM in the drinking water has an inhibitory effect on the H. pylori infection in vivo as observed by the decrease of the H. pylori antigen-specific antibody response.

B Histopathological Analysis and Grading of Gastric Lesions.

In these conditions, a lower level of PGE₂ in the sera was also observed (FIG. 9A), likely related to an anti-inflammatory action of DHA.

As previously reported in the mouse model of infection (ref Touati 2003), the analysis of the gastric inflammatory lesions of the infected mice, showed the presence of infiltrates of PMN and plasmocytes in the infected mucosa with score grading slightly decreased in the presence of DHA only observed in the fundus (FIG. 9B and FIG. 9C).

Concerning to histological lesions, the presence of DHA decreases the severity of hyperplasic lesions with less architectural atypies that were observed in SS1-INS-GAS infected mice.

### REFERENCES

1—Graham D Y, Agrawal N M, Campbell D R, et al. Ulcer prevention in long-term users of onsteroidal anti-inflammatory drugs; results of a double-blind, randomized, multicenter, active and placebo controlled study of misoprostol vs lansoprazole. Arch Intern Med 2002; 162:169-75.

2—Peek RM, Blaser M J. Helicobacter pylori and gastrointestinal tract adenocarcinomas. Nat Rev Cancer 2002, 2:28-37).

3—Boyanova L, Spassova Z, Krastev Z, et al. Characteristics and trends in macrolide resistance among Helicobacter pylori strains isolated in Bulgaria over four years. Diagn Microbiol Infect Dis 1999; 34:309-13.

4—Bago J, Halle Z B, Strinic D, et al. The impact of primary antibiotic resistance on the efficacy of ranitidine bismuth citrate- vs. omeprazole-based one-week triple therapies in H. pylori eradication—a randomised controlled trial. Wien Klin Wochenschr 2002; 114:448-53.

5—Mégraud F. Surveillance de la resistance de Helicobacter pylori aux antibiotiques. In: Surveillance nationale des maladies infectieuses 1998-2000. St Maurice, France: Institut de Veille Sanitaire, 2003: 327-9.

6—Wolle K, Leodolter A, Malfertheiner P, et al. Antibiotic susceptibility of Helicobacter pylori in Germany: stable primary resistance from 1995 to 2000. J Med Microbiol 2002; 51:705-9.

7—Dammann H G, Folsch U R, Hahn E G, et al. Eradication of H. pylori. With pantoprazole, clarithromycin, and metronidazole in duodenal ulcer patients: a head-to-head comparison between two regimens of different duration, Helicobacter, 2000; 5:41-51.

8—Toracchio S, Marzio L. Primary and secondary antibiotic resistance of Helicobacter pylori strains isolated in central Italy during the years 1998-2002. Dig Liver Dis 2003; 35:541-5.

9—Pilotto A, Rassu M, Leandro G, et al. Prevalence of Helicobacter pylori resistance to antibiotics in Northeast Italy: a multicentre study. GISU. Interdisciplinary Group for the Study of Ulcer. Dig Liver Dis 2000; 32:763-8.

10—Debets-Ossenkopp Y J, Herscheid A, Pot R G J, et al. Prevalence of Helicobacter pylori resistance to metronidazole, clarithromycin, amoxicillin, tetracycline and trovafloxacin in The Netherlands. J Antimicrob Chemother 1999; 43:511-15.

11—Cabrita J, Oleastro M, Matos R, et al. Features and trends in Helicobacter pylori antibiotic resistance in Lisbon area, Portugal (1990-1999). J Antimicrob Chemother 2000; 46:1029-31.

12—Cuchi Burgos E, Forne Bardera M, Quintana Riera S, et al. Evolution of the sensitivity of 235 strains of Helicobacter pylori from 1995 to 1998 and impact of antibiotic treatment Enferm Infecc Microbiol Clin 2002; 20:157-60.

13—Megraud F, Lehn N, Lind T, et al. Antimicrobial susceptibility testing of Helicobacter pylori in a large multicentre trial: the MACH 2 study. Antimicrob Agents Chemother 1999; 43:2747-52.

14—Parsons H K, Carter M J, Sanders D S, et al. Helicobacter pylori antimicrobial resistance in the United Kingdom: the effect of age, sex and socio-economic status. Aliment Pharmacol Ther 2001; 15:1473-8.

15—Teare L, Peters T, Saverymuttu S, et al. Antibiotic resistance in Helicobacter pylori. Lancet 1999; 353:242

16—Osato M S, Reddy R, Reddy S G, et al. Pattern of primary resistance of Helicobacter pylori to metronidazole or clarithromycin in the United States. Arch Intern Med 2001; 161:1217-20.

17—Laine L, Fennerty M B, Osato M, et al. Esomeprazole-based Helicobacter pylori eradication therapy and the effect of antibiotic resistance: results of three US multicentre, double-blind trials. Am J Gastroenterol 2000; 95:3393-8.

18—Laine L, Hunt R, E I-Zimaity H, et al. Bismuth-based quadruple therapy using a single capsule of bismuth biskal citrate, metronidazole, and tetracycline given with omeprazole versus omeprazole, amoxicillin, and clarithromycin for eradication of Helicobacter pylori in duodenal ulcer patients: a prospective, randomized, multicentre, North American trial. Am J Gastroenterol 2003; 98:562-7.

19—Megraud F. H pylori antibiotic resistance: prevalence, importance and advances in testing. Gut 2004; 53:1374-1384.

20—Glupczynski Y, Megraud F, Lopez-Brea M, et al. European multicenter survey of in vitro antimicrobial resistance in Helicobacter pylori. Eur J Clin Microbiol Infect Dis 2000; 11:820-3.

21—Gerhard M, Rad R, Prinz C, Naumann M. Pathogenesis of Helicobacter pylori infection. Helicobacter 2002; 7:17-23.

22—Vakil N, Mégraud F. Reviews in basic and clinical gastroenterology. Gastroenterology 2007; 133:985-1001.

23—Hoffelner H, Rieder G, Haas R. Helicobacter pylori vaccine development: Optimization of strategies and importance of challenging strain and animal model. Int J Med Microbiol. 2008; 298(1-2):151-9.

24—Bergonzelli G, Donnicola D, Porta N, Corthésy-Thelauz I. Essentials oils as components of a diet-based approach to management of Helicobacter infection. Antimicrobial Agents and Chemotherapy, 2003; 47(10):3240-6.

25—Marino S, Borbone N, Gala F, Zollo F, Fico G, et al. New constituents of sweet capsicum annum L. fruits and evaluation of their biological activity. Agriculture and food. Chemistry 2006; 54:7508-16.

26—Petschow B, Batema R, Ford L. Susceptibility of Helicobacter pylori to bactericidal properties of medium chain monoglycerides and free fatty acids. Antimicrobial agents and Chemotherapy, 1996; 40(2):302-6.

27—Tamawski A, Hollander D, Gergely H. Protection of the gastric mucosa by linoleic acid—a nutrient essential fatty acid. Clin Invest Med 1987; 10: 132-5.

28—Thompson L, Cockayne A, Spiller. Inhibition effect of polyunsaturated fatty acids on the growth of helicobacter pylori: a possible explanation of the effect of diet on peptic ulceration. Gut 1994; 35:1557-61.

29—Knapp R, Melly A. Bactericidal effects of polyunsaturated fatty acids. Journal Infect Dis 1986; 154:84-94.

30—Das U. N., Devi G. R, Rao K. P., Ran M. S. Prostaglandins can modify gamma-radiation and chemical-induced cytotoxicity and genetic damage both in vitro and in vivo. Prostaglandins 1989; 38: 689-716.

31—Wyss O., Ludwig B. J., Joiner R. R. The fungistatic and fungicidal action of fatty acids and related compounds. Arch Biochem 1945; 7: 415-425.

32—Lees A. M., Korn E. D. Metabolism of unsaturated fatty acids in protozoa. Biochemistry 1966; 15:1475-1481.

33—Das U. N. Antibiotic-like action of essential fatty acids. Can Med Assoc J 1985; 123: 1350.

34—Sands J. A. Inactivation and inhibition of replication of the enveloped bacteriophage 06 by fatty acids. Antimicrob Agents Chemother 1977; 12: 523-530.

35—Lee A, O'Rourke J, Ungria M, Robertson D, Daskalopoulos G, Dixon M. A standardized mouse model of Helicobacter pylori infection: introducing the Sydney strain. Gastroenterology, 1997, 112: 1386-1397

36—Israel D, Salama N, Arnold C N, Moss S F, Ando T, Wirth H P, Tham K T, Camorlinga M, Blaser M J, Falkow S and Peek R M (2001) J. Clin. Invest, 107: 611-620

37—US2007/0021508

38—US 2003/0032674

39—JP 10-130161

40—Wang T C, Bonner-Weir S, Oates P S, Chulak M B, Simon B, Merlino, G T, Schmidt E V and Brand S J (1993) Pancreatic gastrin stimulates islate differenciation of transforming Growth Factor alpha-induced Ductular Precursor cells, The J of Clinical Investigation, 92: 1349-1356

41—Drago et al Effects of three different fish oil formulations on Helicobacter pylori growth and viability: in vitro study: Journal of Chemotherapy 1999; 11(3):207-10.

42—Frieri G et al. Polyunsaturated fatty acid dietary supplementation: an adjuvant approach to treatment of Helicobacter pylori infection. Nutrition research 2000; 20(7):907-16.

43—Fox J. G et al, Helicobacter pylori-associated gastric cancer in INS-GAS mice is gender specific. Cancer Research, 2003; 63:942-950.

44—European Helicobacter pylori Study Group. Current European concepts in the management of Helicobacter pylori infection. The Maastricht consensus report. Gut 1997; 41:8-13.

45—The report of the Digestive Health Initiative International Update on Helicobacter pylori. Gastroenterology 1997; 113:S4-5].

46—Lam S K, Talley N J. Report of the 1997 Asia Pacific Consensus Conference on the management of Helicobacter pylori infection. J Gastroenterol Hepatol 1998; 13:1-12.

47—Hunt R H, Fallone C A, Thomson A B R. Canadian Helicobacter pylori Consensus Conference update: infections in adults. Can J Gastroenterol 1999; 13:213-17.].

48—Coelho L G, Leon-Barua R, Quigley E M M, et al. Latin-American consensus conference on Helicobacter pylori infection. Am J Gastroenterol 2000; 95:2688-91.

49—Touati E, Michel V, Thiberge J M, Wuscher N, Huerre M and Labigne A, 2003, Chronic Helicobacter pylori infection induce gastric mutations in mice, Gastroenterology, 124, 1408-1419

50—Eaton K A, Radin M J, Krakowa S. An animal model of gastric ulcer due to bacterial gastritis in mice. Vet Pathol 1995; 32:489-497

51—Ferrero et al, 1998; Infection Immunity 66: 1349-1355.

52—Lee C-W, Rickman B, Rogers A. B., Ge Z, Wang T C and Fox J G, 2008, Helicobacter pylori eradication prevents progression of gastric cancer in hypergastrinemics INS-GAS mice. Cancer Research, 68: 3540-3548.

53—van Zanten S J, Kolesnikow T, Leung V, O'Rourke J L, Lee A. Gastric transitional zones, and areas where Helicobacter treatment fails: results of a treatment trial using Sydney strain mouse model. Antimicrob Agents Chemother 2003; 47:2249-55.

1. A method of preventing, modulating or curing in a mammal, the infections with Helicobacter pylori or the symptoms associated with H. pylori infection(s) or related diseases, and/or improving the clinical condition of a mammal infected with H. pylori or affected by a related disease, comprising administering to a mammal in need thereof an effective amount of one or more DHA related compound selected from the group comprising the docosahexaenoic acid (DHA), pharmaceutically acceptable salts thereof, pharmaceutically acceptable esters or derivatives thereof, as well as pharmaceutically acceptable precursors or prodrugs thereof and metabolites thereof and their mixtures.

2. The method of claim 1, wherein said infection is chronical infection with H. pylori or its recurrence or its relapse.

3. The method of claim 1, wherein said infection is chronical infection with H. pylori.

4. The method of claim 1, wherein the DHA related compound prevents, decreases, alleviates or abolishes chronic inflammation resulting from H. pylori infection in a mammal.

5. The method of claim 1, wherein the related disease is selected from the group consisting of active gastritis, chronic atrophic gastritis, gastric ulcer, duodenal ulcer, peptic ulcer disease, gastric cancer, peptic esophagitis, gastric adeno carcinoma or mucosa-associated lymphoid tissue lymphomas.

6. The method of claim 1, wherein the DHA related compound is docosahexaenoic acid in its free acidic form.

7. The method of claim 1, wherein the dosage ranges from 1 mg/kg/day to 10 mg/kg/day, preferably 1.8 mg/kg/day to 6 mg/kg/day of DHA.

8. The method of claim 1, wherein the dosage applied to a man with an average body weight of 70 kg is between 100 and 500 mg, preferably 120 to 420 mg.

9. The method of claim 1, wherein the concentration of DHA in the stomach lumen is of between 25 and 100 mM, preferably between 40 and 60 mM, more preferably about 50 mM.

10. The method of claim 1, wherein the DHA related compound is formulated as a composition comprising in addition to said DHA related compound, suitable excipients such as diluents, stabilizers, solvents, surfactants, buffers, carriers, preservatives or adjuvants.

11. The method of claim 1, wherein said a therapeutically effective amount is administered orally or oesophagically or gastrically.

12. The method of claim 1, wherein said a therapeutically effective amount is administered orally.

13. The method of claim 1, wherein the dosage form is selected from the group consisting of soft or hard capsules, tablets, powders, granules, pastes, syrups, solutions, W/O or O/W emulsions, suspensions, liposome formulations, microcapsules, nanocapsules, long release formulations, particularly those adapted for stomach-coating medication or gastrointestinal protectants.

14. The method of claim 1, wherein said therapeutically effective amount is administered in a protocol encompassing administration of one or more further therapeutically active substance(s) selected from the group consisting of gastro-intestinal protectants, proton pump inhibitors, H2 blockers, antibiotics, anti-inflammatory substances, anti-tumor compounds, particularly amoxicillin, clarithromycin, tetracycline, rifabutin, fluoroquinolones, metronidazole.

15. DHA used as an adjuvant or co-adjuvant of one or more pharmaceutically active compound in a method of preventing, modulating or curing in a mammal, an infection with Helicobacter pylori or the symptoms associated with H. pylori infection or related diseases, and/or improving the clinical condition of a mammal infected with H. pylori, comprising administering to a mammal in need thereof an effective amount of said active compound and of said adjuvant or co-adjuvant.
